(19) United States
(12) Patent Application Publication
(10) Pub. No.: US 2023/0248566 A1
(43) Pub. Date: Aug. 10, 2023

(54) ORAL APPLIANCE AND METHOD FOR TREATING SLEEP DISORDERS

(71) Applicants: Jose VENEGAS, West Roxbury, MA (US); United States Government as Represented by the Department of Veterans Affairs, Washington, DC (US); MASSACHUSETTS INSTITUTE OF TECHNOLOGY, Cambridge, MA (US)

(72) Inventors: RAVI RASALINGAM, Boston, MA (US); TARSHA WARD, Lowell, MA (US); JOSE VENEGAS, Boston, MA (US); Ellen T. Roche, Cambridge, MA (US)

(21) Appl. No.: 18/010,617

(22) PCT Filed: Jun. 17, 2021
(86) PCT No.: PCT/US2021/037806

§ 371 Date: Dec. 15, 2022

Related U.S. Application Data
(60) Provisional application No. 63/040,700, filed on Jun. 18, 2020.

(51) Int. Cl. A61F 5/56; A61M 16/00

(52) U.S. Cl. CPC A61F 5/566; A61M 16/0009

(57) ABSTRACT

An oral appliance and method for treating a sleep disorder in a subject are disclosed. The appliance includes, among other elements, a palatal overlay element that engages a portion of the dorsal surface of the subject's tongue, stabilizing the tongue in a superior direction toward the hard palate. The method generally involves stabilizing the dorsal surface of a subject's tongue in a superior direction toward the subject's hard palate.

### CROSS-REFERENCE TO RELATED APPLICATION

This application claims priority to and the benefit of the filing date of U.S. Provisional Application No. 63/040,700, filed Jun. 18, 2020, the entirety of which is hereby incorporated by reference herein.

### FIELD

This application relates generally to an oral appliance and method for treating a sleep disorder in a subject by stabilizing the tongue in a superior direction toward the subject's hard palate.

### BACKGROUND

A variety of sleep disorders involve mouth physiology. An example is obstructive Sleep Apnea (OSA), which is a common, under-diagnosed disorder that causes repetitive closure of the back of the throat. OSA interferes with breathing and results in poor sleep and stress to the body. The disorder has been associated with daytime sleepiness, reduced cognitive function, arrhythmias, high blood pressure, stroke, and premature death. Closure of the back of the throat occurs when the muscle tone of the throat muscles reduces during sleep. This typically results when the tongue slides backwards and obstructs airflow through the nose and mouth.

A common therapy for OSA involves the use of a continuous positive airway pressure (CPAP) device. A CPAP device keeps a patient's airway open during sleep by forcing air through a tight-fitting mask that covers the mouth and/or nose. For many patients, a CPAP device is uncomfortable, cumbersome, and presents a significant change to the normal sleep environment. Consequently, 50% of OSA patients refuse CPAP treatment, and 30% of patients do not tolerate it. For patients with OSA who cannot tolerate or have difficulty consistently using CPAP treatment, sleep without breathing obstruction is difficult.

Various oral prostheses are also used for OSA treatment. Two common prostheses are the mandibular advancement device (MAD) and the tongue retaining device (TRD). A MAD is designed to mechanically move the entire jaw forward, while a TRD pulls the tongue out of the mouth. Like the CPAP device, however, MADs and TRDs are poorly tolerated. These devices may also cause persistent jaw and tongue pain, and even dental or facial deformity.

There is currently no effective therapy that a majority of OSA patients can tolerate on a consistent basis. Thus, there is a need for an alternative therapy that allows OSA patients to breath without obstruction and minimizes discomfort and disturbance of sleep. This need and others are met by the oral appliance and method described below.

### SUMMARY

Disclosed herein, in one aspect, is an oral appliance for treating a sleep disorder in a subject. The oral appliance can comprise a U-shaped element, a palatal overlay element, and a port configured to apply negative pressure to the appliance such that, when in use, the appliance can stabilize a subject's tongue by engaging the tongue upward in a superior direction toward the subject's hard palate.

The U-shaped element can have an outer side wall, an inner side wall, and at least one biting surface, with the outer side wall, the inner side wall, and the at least one biting surface together defining at least one channel configured to receive at least one upper tooth of the subject.

The palatal overlay element can be contiguous with the U-shaped element and configured to conform to at least a portion of the subject's hard palate. The palatal overlay element can have an interior surface and an exterior surface and define a plurality of perforations (e.g., openings) extending through the palatal overlay element between the interior and exterior surfaces. The plurality of perforations can be oriented in an inferior direction along a longitudinal axis of the subject.

The port can be in fluid communication with the plurality of perforations of the palatal overlay element and configured to provide negative pressure to the plurality of perforations of the palatal overlay element.

The exterior surface of the palatal overlay element can be configured to engage at least a portion of the dorsal surface of the subject's tongue in response to application of negative pressure through the plurality of perforations of the palatal overlay element.

Also disclosed herein, in another aspect, is a method for treating a sleep disorder in a subject. The method can comprise stabilizing the subject's tongue in a superior direction toward at least a portion of the subject's hard palate, e.g., through the application of negative pressure or through an adhesion mechanism such as a wet-tolerant adhesive. The oral appliance can be used to perform the method in various aspects, but the method is not limited to any particular device or appliance.

Additional advantages of the disclosed appliance and method will be set forth in part in the description which follows, and in part will be understood from the description, or may be learned by practice of the disclosed oral appliance and method. The advantages of the disclosed appliance and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed oral appliance and method and together with the description, serve to explain the principles of the disclosed appliance and method.

FIG. 1 depicts an embodiment of an oral appliance in which the port in fluid communication with the plurality of perforations is positioned near an anterior region of the oral appliance.

FIG. 2 depicts an embodiment of an oral appliance in which the port in fluid communication with the plurality of perforations is positioned near a posterior region of the oral appliance.

FIG. 3 depicts a subject using an embodiment of an oral appliance during sleep.

### DETAILED DESCRIPTION

The disclosed oral appliance and method may be understood more readily by reference to the following detailed description of particular embodiments and the examples included therein and to the Figures and their previous and following description.

### A. Definitions

It is to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a port” includes a plurality of such ports, and reference to “the port” is a reference to one or ports and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

As used herein, the term “hard palate” refers to the thin bony plate made up of bones of the facial skeleton, located in the roof of a subject's oral cavity, closest to the opening of the mouth. The hard palate is distinguished from the “soft palate” in that the soft palate does not contain any bone but rather is a fleshy area farther into the subject's oral cavity, ending at the uvula (the fleshy extension at the back of the oral cavity that hangs above the throat).

The term “molar tooth” as used herein refers to an upper (maxillary) molar tooth, including the maxillary first molar, maxillary second molar, and maxillary third molar (also known as the wisdom tooth). Molar teeth have either four or five cusps.

The term “incisor tooth” as used herein refers to an upper (maxillary) incisor tooth, including the two maxillary central incisors (closest to the center of the mouth) and the two maxillary lateral incisors (beside the maxillary central incisor).

As used herein, the term “thermoplastic” refers to a polymer material that is pliable or moldable at a certain elevated temperature and solidifies upon cooling.

The term “silicone” as used herein refers to a polymer material that includes any compound made up of repeating units of siloxane, which is a chain of alternating silicon and oxygen atoms, combined with carbon, hydrogen, and sometimes other elements.

As used herein, the term “acrylic” refers to a glassy thermoplastic polymer material made from acrylic acid.

The term “tacky polymer” as used herein refers to any polymer material that is slightly sticky or adhesive at or near room temperature.

As used herein, a “condition associated with mouth physiology” means any condition contributing to a sleep disorder involving the misalignment or misplacement of the various tissues and bones present in the cavity of the mouth and throat. Non-limiting examples include obstructive sleep apnea, mouth breathing, snoring, teeth grinding, xerostomia, halitosis, hyper-salivation, tardive dyskinesia, and the like.

As used herein, “conform to” should be understood to mean either (a) deform to match the shape of; or (b) have a shape that is complementary to a stated surface or structure.

Unless defined otherwise, all technical and scientific terms used herein have the same meanings as commonly understood by one of skill in the art to which the disclosed appliance and method belong. Although any oral appliance and method similar or equivalent to those described herein can be used in the practice or testing of the present appliance and method, the particularly useful appliances and methods are as described.

Throughout the description and claims of this specification, the word “comprise” and variations of the word, such as “comprising” and “comprises,” means “including but not limited to,” and is not intended to exclude, for example, other elements, components, integers or steps. In particular, in methods stated as comprising one or more steps or operations, it is specifically contemplated that each step comprises what is listed (unless that step includes a limiting term such as “consisting of”), meaning that each step is not intended to exclude, for example, other elements, components, integers or steps that are not listed in the step.

### B. Oral Appliance

Disclosed herein is an oral appliance for treating a sleep disorder in a subject (e.g., a human) that can include, among other elements, a palatal overlay element through which suction (negative pressure) can be applied such that the palatal element engages the dorsal surface of the subject's tongue, to stabilize the tongue in a superior direction toward the hard palate. According to one aspect, the oral appliance can be designed to be custom fit to individual subjects and to use gentle suction applied through the palatal element to prevent throat obstruction and airway closure during sleep.

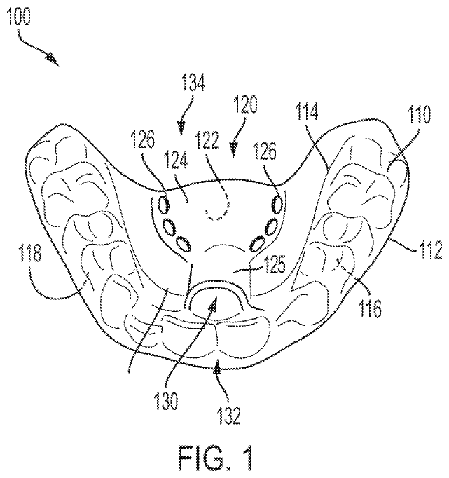

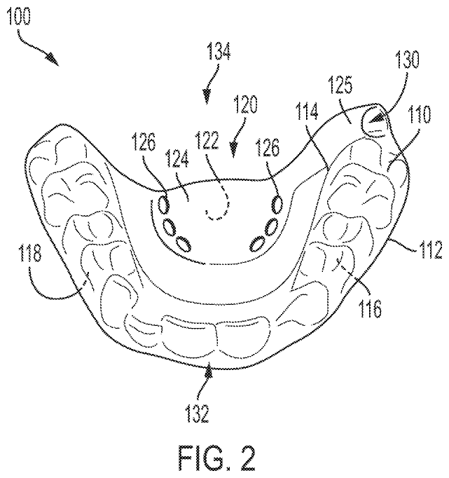

Referring to FIGS. 1 and 2, the oral appliance 100 can have a U-shaped element 110, a palatal overlay element 120, and a port 130 through which suction (negative pressure) can be applied. The U-shaped element 110 can comprise an outer side wall 112, an inner side wall 114, and at least one biting surface 116. Together, the outer side wall 112, inner side wall 114, and at least one biting surface 116 define a channel 118 configured to receive at least one upper (maxillary) tooth of a subject. According to one aspect, the channel 118 is configured to receive most or all of a subject's upper teeth. Such a configuration can be made, for example, from a mold of the subject's upper teeth, based on an optical image obtained from a scan of the subject's oral cavity, or through other methods known in the art.

In further aspects, the oral appliance can have any suitable shape such that the appliance fits within the cavity of a subject's mouth. In one aspect, for example, the oral appliance can be shaped so as to provide conformity with at least a portion of the subject's hard palate. An appliance of any suitable shape and form can form a non-permanent attachment between the dorsal surface of the subject's tongue and the subject's hard palate, to stabilize the tongue in a superior direction toward the hard palate.

Referring again to FIGS. 1 and 2, the palatal overlay element 120 can be contiguous with the U-shaped element 110. For example, in some optional aspects, the palatal overlay element can extend about and between opposing portions the inner side wall 114 of the U-shaped element. Thus, in some optional aspects, the palatal overlay element 120 and U-shaped element 110 can meet at a continuous interface that extends along the entire, or substantially the entire, inner wall 114. According to one aspect, the palatal overlay element 120 and U-shaped element 110 can be a monolithic body, or a single structure. In other aspects, the palatal overlay element 120 and U-shaped element 110 can be contiguous but not monolithic. That is, palatal overlay element 120 and U-shaped element 110 can be made separately and combined or formed into a composite material or assembly.

The palatal overlay element 120 can comprise an interior surface 122, which faces the subject's hard palate, and an opposing exterior surface 124, which faces the subject's tongue. The interior surface 122 can define one or more hollow chambers 125 through which suction (negative pressure) can be applied. Optionally, during use of the oral appliance 100 as further disclosed herein, the one or more hollow chambers 125 defined by the interior surface 122 of the palatal overlay element 120 can further be defined in part by the subject's hard palate. Specifically, according to one aspect, negative pressure can be applied to the dorsal surface of a subject's tongue through a plurality of perforations (e.g., openings) 126 that extend through the palatal overlay element 120 between the interior 122 and exterior 124 surfaces. The plurality of perforations 126 can be oriented in an inferior direction along a longitudinal axis of the subject. Thus, in one aspect, the plurality of perforations 126 can be oriented such that the perforations engage the dorsal surface of the subject's tongue when negative pressure is applied through the palatal overlay element 120.

According to one aspect, the plurality of perforations 126 can be designed to allow for gentle suction (negative pressure) on the dorsal surface of the tongue at different locations on the tongue surface, so as to minimize any one part of the tongue from getting irritated or injured during use. Thus, in some aspects, the number perforations in the plurality of perforations 120 can depend on variables such as the surface area of a subject's hard palate, the weight of the tongue, and the amount of negative pressure required to engage and pull the tongue in a superior direction toward the hard palate. In the exemplary embodiments shown in FIGS. 1 and 2, the plurality of perforations 126 comprises six perforations. According to other aspects, the plurality of perforations 126 can have more than two perforations. In further aspects, the plurality of perforations 126 can include two to twelve perforations. In still further aspects, the plurality of perforations 126 can have four to eight perforations, or four to six perforations. In some optional aspects, the perforations can be circular, square, rectangular, oblong, or any other suitable shape. In some optional aspects, each of the perforations can define an area therethrough of from one square millimeter to twenty square millimeters (e.g., optionally, about two square millimeters, or about five square millimeters). In some optional aspects, the perforations 126 can collectively define an area therethrough (i.e., a sum of the areas of all of the perforations 126) of at least eight square millimeters, at least 20 square millimeters, at least 40 square millimeters, or less than 100 square millimeters.

Optionally, as shown in FIGS. 1-2, it is contemplated that the plurality of perforations 126 can be arranged as two spaced clusters of perforations positioned on opposing lateral edges of the palatal overlay element. In exemplary aspects, the number of perforations in each cluster can be equal. In other aspects, the number of perforations in each cluster can be different. In still further aspects, it is contemplated that the perforations within each cluster can be substantially evenly spaced (measured from center-point to center-point) moving along a respective lateral edge of the palatal overlay element. Alternatively, it is contemplated that the perforations within each cluster can be unevenly spaced. In further exemplary aspects, it is contemplated that the plurality of perforations can comprise rows and/or columns of perforations that extend across the width and/or length of the palatal overlay element. In these aspects, it is contemplated that even or uneven spacing (center point to center point) can be used within each row and/or column. Optionally, when rows are used, it is contemplated that the perforations of one row can be offset relative to the perforations of sequential rows.

The port 130 can be in fluid communication with the plurality of perforations 126. According to one aspect, the port 130 can be configured to provide suction (negative pressure) to the plurality of perforations 126 of the palatal overlay element 120. In exemplary aspects, the port 130 can comprise or be in fluid communication with a conduit (e.g., the one or more hollow chambers 125) that extends between an inlet/outlet of the port and the palatal overlay element 120.

In one aspect, such as shown in FIG. 1, the port 130 can be positioned near an anterior (front) region 132 of the oral appliance 100. Thus, for example, the port 130 can be positioned near an incisor tooth of the subject when the oral appliance 100 is inserted into the subject's oral cavity (e.g., the port 130 is at a location near the portion of the U-shaped element 110 that is configured to receive said incisor). In the exemplary embodiment shown in FIG. 1, the port 130 is positioned near the maxillary central incisors (the incisors closest to the center of the mouth) (e.g., the port 130 is at a location near the portion of the U-shaped element 110 that is configured to receive said incisors). In other aspects, the port 130 can be positioned near a maxillary lateral incisor (the incisors next to the maxillary central incisors) (e.g., the port 130 is at a location near the portion of the U-shaped element 110 that is configured to receive said incisor).

In another aspect, such as shown in FIG. 2, the port 130 can be positioned at or near a posterior (rear) region 134 of the oral appliance 100. Thus, for example, the port 130 can be positioned near a molar tooth of the subject when the oral appliance 100 is inserted into the subject's oral cavity (e.g., the port 130 is at a location near the portion of the U-shaped element 110 that is configured to receive the molar tooth). In some aspects, the port 130 can be positioned near the maxillary first molar, the maxillary second molar, or the maxillary third molar (also known as the wisdom tooth) when the oral appliance 100 is inserted into the subject's oral cavity (e.g., the port 130 is at a location near the portion of the U-shaped element 110 that is configured to receive the said tooth). In a further aspect, the port 130 can be positioned near the subject's last upper molar, often the maxillary second molar in subjects without one or more wisdom teeth (e.g., the port 130 is at a location near the portion of the U-shaped element 110 that is configured to receive the said tooth). Such an exemplary embodiment allows for the subject to close his or her mouth when the oral appliance 100 is inserted into the oral cavity and connected to a source of negative pressure through a tube.

As described herein, in some aspects, “near” a tooth can be understood to mean within about 3 cm, within about 2 cm, within about 1 cm, or within about 0.5 cm of said tooth. Optionally, in some aspects, the anterior region 132 and the posterior region 134 can be separated by an imaginary line that divides the length of the oral appliance in half, with the front half (toward the front of the subject) being the anterior region and the back half (toward the back or rear of the subject) being the posterior region. In other optional aspects, the anterior region 132 and the posterior region 134 can refer to respective thirds of the oral appliance that are closest to the front-most or rear-most surfaces/edges of the appliance.

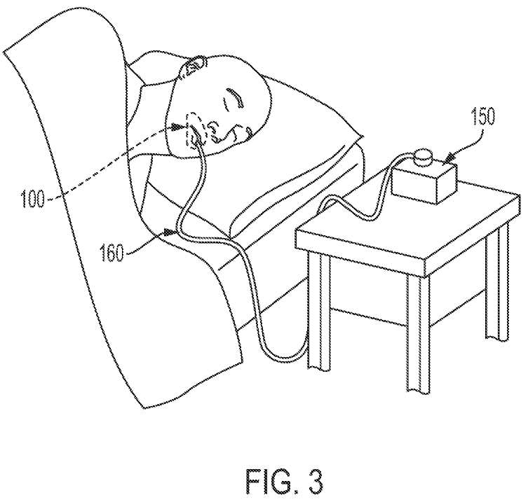

With reference to FIG. 3, for example, the oral appliance 100 can comprise a source of negative pressure 150 in fluid communication with the port 130. According to one aspect, the oral appliance 100 can comprise a tube 160 (e.g., any suitable conduit) that provides fluid communication between the source of negative pressure 150 and the port 130 shown in FIGS. 1 and 2. In some optional aspects, at least one coupling element can couple the tube 160 to the port 130. For example, in some optional aspects, the port 130 can define a first coupling element that is configured to couple to a second coupling element of the tube 160. The first and second coupling elements can optionally comprise, for example, complementary male and female threads or other tube/hose couplings. In further aspects, the port 130 can comprise a tube fitting (e.g., a quick disconnect or barbed coupling) that is configured to receive the tube 160. In yet further aspects, any suitable coupling elements between the tube 160 and the port 130 (e.g., barbed fittings) are contemplated. In still further aspects, the tube 160 can be integrally formed with the port 130. In one aspect, the source of negative pressure 150 can be a vacuum pump. In a further aspect, the source of negative pressure 150 can be a micropump, e.g., a pump that can be as small as a few inches in length or in some aspects, even smaller. Alternatively, the source of negative pressure can be a syringe that can be operated manually by a subject, e.g., by pulling or pushing the plunger of the syringe (optionally, repeating periodically) to create a vacuum capable of applying negative pressure to the plurality of perforations of the palatal overlay element.

In still further aspects, the source of negative pressure 150 can be configured to generate a suction pressure of at least −5 cm H₂O. The source of negative pressure 150, for example, can be configured to generate a suction pressure of between about −5 cm H₂O to about −20 cm H₂O, or, in other aspects, between about −5 cm H₂O to about −10 cm H₂O. The amount of suction pressure the source of negative pressure 150 can be designed to provide can vary depending on the application. Thus, in some aspects, the source of negative pressure 150 can be configured to apply gentle or a comfortable level of suction to the dorsal surface of a subject's tongue.

Tube 160 can be any suitably-sized tube formed from materials known in the art. According to one aspect, tube 160 can have a diameter (e.g., an outer diameter) in the millimeter range, e.g., 1-5 mm, which can be a suitable size for use with a micropump. Length of the tube 160 can also vary depending on the type of negative pressure source 150 and where the subject prefers to locate the negative pressure source, e.g., attached or clipped to the subject's body or clothing or placed on a bed-side table or the like. Optionally, tube 160 can be connected to a saliva pot or receptacle to capture any saliva pulled out of the oral cavity by the source of negative pressure.

When in use, the oral appliance can allow for a subject's tongue to be stabilized in a superior direction toward at least a portion of the subject's hard palate. The source of negative pressure can transmit suction through a tube to the palatal overlay element of the oral appliance. Suction can be applied to the dorsal surface of the tongue as suction is transmitted through the plurality of perforations of the palatal overlay element. In some aspects, for example, the oral appliance allows for the tongue to remain in a neutral position in terms of anterior-posterior movement in the oral cavity and avoids the tongue coming out of the mouth during use. The oral appliance, in various aspects, can improve airway patency and nasal breathing during sleep. Stabilizing the tongue in a superior direction toward the hard palate can also treat or alleviate symptoms associated with a variety of sleep disorders involving mouth physiology, including without limitation obstructive sleep apnea, mouth breathing, snoring, teeth grinding, xerostomia, halitosis, hyper-salivation, tardive dyskinesia, and the like.

According to one aspect, the oral appliance, U-shaped element, and/or the palatal overlay element can comprise or be formed from any material suitable for insertion into a subject's oral cavity. The materials chosen for the oral appliance can be selected to maximize comfort for subject's oral cavity. Similarly, the materials can be selected to provide a non-permanent attachment (e.g., through an adhesive mechanism) of at least a portion of the oral appliance to the dorsal surface of the subject's tongue. Non-limiting examples of suitable materials include without limitation various thermoplastics, silicones, acrylics, biocompatible materials (e.g., Formlabs; RS-F2-DLCL-01), tacky polymers (which can help promote engagement of the palatal overlay element with the tongue), or a combination thereof. Tacky polymers can include nano- or macroscopic-sized architectural features such as biomimetic wet/dry adhesion materials that can facilitate adherence of the tongue to the palatal overlay element and provide a mechanism for manipulating the tongue muscle to hold it in place while the subject sleeps.

In one aspect, for example, at least a portion of the oral appliance, e.g., the palatal overlay element, can be formed from or comprise a wet-tolerant adhesive on at least a portion of a surface thereof. An example can be the adhesive described in Baik, S., Kim, D., Park, Y. et al. “A wet-tolerant adhesive patch inspired by protuberances in suction cups of octopi.” Nature 546,396-400 (2017), which is hereby incorporated by reference herein in its entirety for its teachings of wet-tolerant adhesives.

Similarly, other suitable wet-tolerant adhesives can include those with hierarchical mushroom-shaped or porous structures that facilitate suction or capillarity, including those described in Dirks, J.-H. & Federle, W. “Fluid-based adhesion in insects—principles and challenges.” Soft Matter 7, 11047-11053 (2011), Xue, L., Kovalev, A., Eichler-Volf, A., Steinhart, M. & Gorb, S. N. “Humidityenhanced wet adhesion on insect-inspired fibrillar adhesive pads.” Nat. Commun. 6, 6621 (2015), Chen, Y., Shih, M.-C., Wu, M.-H., Yang, E.-C. & Chi, K.-J. “Underwater attachment using hairs: the functioning of spatula and sucker setae from male diving beetles.” J. R. Soc. Interface 11, 20140273 (2014), Kovalev, A. E., Varenberg, M. & Gorb, S. N. “Wet versus dry adhesion of biomimetic mushroom-shaped microstructures.” Soft Matter 8, 7560-7566 (2012), each of which is hereby incorporated by reference herein in its entirety for its teachings of wet-tolerant adhesives.

Other suitable wet-tolerant adhesives include those having supramolecular structures comprising nanoparticles, as described in Rose, S. et al. “Nanoparticle solutions as adhesives for gels and biological tissues.” Nature 505,382-385 (2014), which is incorporated herein by reference in its entirety for its teachings of wet-tolerant adhesives. In another aspect, the wet-tolerant adhesive can comprise a chemistry-based attractant that uses various protein polyelectrolytes, as described in Zhao, Q. et al. “Underwater contact adhesion and microarchitecture in polyelectrolyte complexes actuated by solvent exchange.” Nat. Mater. 15,407-412 (2016), Lee, S.-B., Gonzalez-Cabezas, C., Kim, K.-M., Kim, K-N. & Kuroda, K. “Catecholfunctionalized synthetic polymer as a dental adhesive to contaminated dentin surface for a composite restoration.” Biomacromolecules 16, 2265-2275 (2015), White, J. D. & Wilker, J. J. “Underwater bonding with charged polymer mimics of marine mussel adhesive proteins.” Macromolecules 44,5085-5088 (2011), each of which is hereby incorporated by reference herein in its entirety for its teachings of wet-tolerant adhesives.

Thus, according to one aspect, it is contemplated that an oral appliance can adhere to the dorsal surface of a subject's tongue without the aid of externally-applied negative pressure. By using a wet-tolerant adhesive on at least a portion of a surface of the palatal overlay element, for example, the palatal overlay element of the disclosed appliance can adhere to the dorsal surface of the subject's tongue such that the tongue is stabilized in a superior direction through a non-permanent attachment.

The oral appliance can be made using a variety of techniques. According to one aspect, alginate impressions of a subject's teeth and hard palate can be taken, and a stone cast can be prepared from the impressions. It is contemplated, for example, that by taking an alginate impression of the upper and lower teeth, a bite profile for the oral appliance can be modeled and generated to match the subject's bite profile. Next, the dental impressions can be embedded in a stone cast material (e.g., Perfect Cast; B017N286R6). A dental forming machine (e.g., Buffalo; 84500) can be used to limit holes or cracks in the stone cast. The stone impression can be scanned using a three-dimensional scanner (e.g. iPhone app Scandy-pro-3D-scanner) to create an initial computer image that is exported as a stereolithography (STL) file. The STL file can then be imported into software known in the dental arts, such as computer-aided design (CAD) software (e.g., Mimics Innovation Suite MIS 22). The initial computer design can then be modified to incorporate various custom design elements of the palatal overlay element, including for example, the perforations, dental overlay of missing or chipped teeth, and the height of the top of the oral appliance to the palate. The custom oral appliance can then be three-dimensionally printed (e.g., Objet Connex 500 printer; Stratasys), and final modifications to the appliance can be made. A similar method can be used in which instead of alginate impressions, an intraoral scanner (e.g., 3Shape trios) can be used to retrieve a custom scan of the subject's oral cavity. The image can be projected as an initial computer design and modified to incorporate design elements of the palatal overlay element.

Although described herein as a U-shaped element, it is contemplated that element 110 can have any shape that is complementary to at least a portion of the teeth of a subject and stabilizes the palatal overlay element in a suitable position for functioning as disclosed herein. For example, it is contemplated that element 110 can have lateral (side) sections while omitting a front section such that at least a portion of the front teeth of the subject are not received within element 110, while the remaining teeth of the subject are received within the side sections.

### C. Method for Treating Sleep Disorder

Also disclosed herein is a method of treating a sleep disorder in a subject. According to one aspect, the method can comprise stabilizing the tongue in a superior direction toward at least a portion of the subject's hard palate. For example, the tongue can be stabilized by applying negative pressure to the subject's oral cavity in a direction generally perpendicular to at least a portion of a dorsal surface of the subject's tongue. According to another aspect, the tongue can be stabilized in a superior direction through a non-permanent attachment of the dorsal surface of the tongue to an oral appliance, for example an oral appliance comprising a wet-tolerant adhesive on a least a portion of a surface thereof. It will be understood that the disclosed method can be performed with the disclosed appliance, but the method is not limited to any particular appliance.

When the method comprises applying negative pressure to the tongue, the location on the dorsal surface of the subject's tongue to which negative pressure is applied can vary. In one aspect, for example, negative pressure can be applied to the subject's oral cavity in a direction generally perpendicular to a medial region of the tongue. In another aspect, negative pressure can be applied to the subject's oral cavity in a direction generally perpendicular to an anterior region of the tongue, that is, closer to the tip of the subject's tongue and the opening of the mouth.

According to one aspect, the method can be useful for pulling and stabilizing the tongue upward toward the hard palate without substantial movement of the tongue forward toward the anterior region of the oral cavity or backward toward the posterior region of the oral cavity. Thus, for example, the subject's tongue can remain inside the oral cavity while being stabilized in a superior direction, e.g., while negative pressure is being applied or while the tongue is non-permanently attached to an oral appliance through a mechanism such as a wet-tolerant adhesive. By stabilizing the tongue upward toward the hard palate, nasal breathing can be promoted, which can be useful in treating a variety of sleep disorders involving mouth physiology, including for example obstructive sleep apnea, mouth breathing, snoring, teeth grinding, xerostomia, halitosis, hyper-salivation, tardive dyskinesia, and the like.

According to another aspect, negative pressure can be applied to a plurality of positions on the dorsal surface of the subject's tongue. By applying negative pressure to a plurality of positions on the tongue, gentle suction can be applied to the tongue at different locations so as to not irritate or injure the tongue while negative pressure is being applied.

The amount negative pressure applied to the subject's tongue can vary. In one aspect, for example, the applied negative pressure is at least about −5 cm H₂O, for example, from about −5 cm H₂O to about −20 cm H₂O, or from about −5 cm H₂O to about −10 cm H₂O.

Although the method is not limited to any particular device or appliance, in one aspect, the subject's tongue can be stabilized in a superior direction through an oral appliance configured to engage at least a portion of the dorsal surface of the subject's tongue, e.g., in response to application of negative pressure or through an adhesion mechanism such as a wet-tolerant adhesive. The oral appliance, in one aspect, can be an embodiment of the oral appliance described above. Thus, for example, the oral appliance can conform to at least one upper tooth, or most or all of the upper teeth of the subject. Similarly, the oral appliance can conform to least a portion of the subject's hard palate.

### D. Exemplary Aspects

In view of the described appliance, method, and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An oral appliance for treating a sleep disorder in a subject, the oral appliance comprising: (a) a U-shaped element having an outer side wall, an inner side wall, and at least one biting surface, the outer side wall, the inner side wall, and the at least one biting surface together defining at least one channel configured to receive at least one upper tooth of the subject; (b) a palatal overlay element contiguous with the U-shaped element and configured to conform to at least a portion of the subject's hard palate, the palatal overlay element having an interior surface and an exterior surface and defining a plurality of perforations extending through the palatal overlay element between the interior and exterior surfaces; (c) a port in fluid communication with the plurality of perforations of the palatal overlay element, wherein the port is configured to communicate negative pressure to the plurality of perforations of the palatal overlay element; wherein the exterior surface of the palatal overlay element is configured to engage at least a portion of a dorsal surface of the subject's tongue in response to application of negative pressure through the plurality of perforations of the palatal overlay element.

Aspect 2: The oral appliance of aspect 1, wherein the U-shaped element and the palatal overlay element are a monolithic body.

Aspect 3: The oral appliance of aspect 1 or aspect 2, wherein the U-shaped element and the palatal overlay element are formed in the shape of a mold taken of at least one upper tooth of the subject and at least a portion of the subject's hard palate.

Aspect 4: The oral appliance of any one of the preceding aspects, wherein the U-shaped element and/or the palatal overlay element comprise(s) a thermoplastic, a silicone, an acrylic, a tacky polymer, or a combination thereof

Aspect 5: The oral appliance of any one of the preceding aspects, wherein the number of perforations is more than two.

Aspect 6: The oral appliance of any one of aspects 1-4, wherein the number of perforations ranges from two to twenty-four.

Aspect 7: The oral appliance of any one of aspects 1-4, wherein the number of perforations ranges from four to eight.

Aspect 8: The oral appliance of any one of aspects 1-4, wherein the number of perforations ranges from four to six.

Aspect 9: The oral appliance of any one of aspects 1-4, wherein the number of perforations is six.

Aspect 10: The oral appliance of any one of the preceding aspects, wherein the port is positioned near a posterior region of the oral appliance.

Aspect 11: The oral appliance of any one of aspects 1-9, wherein the port is positioned near an anterior region of the oral appliance.

Aspect 12: The oral appliance of any one of aspects 1-9, wherein the port is positioned near a molar tooth of the subject when the oral appliance is inserted into the subject's oral cavity.

Aspect 13: The oral appliance of any one of aspects 1-9, wherein the port is positioned near an incisor tooth of the subject when the oral appliance is inserted into the subject's oral cavity.

Aspect 14: The oral appliance of any one of the preceding aspects, further comprising a source of negative pressure in fluid communication with the port.

Aspect 15: The oral appliance of any one of the preceding aspects, further comprising a tube that provides fluid communication between the source of negative pressure and the port.

Aspect 16: The oral appliance of aspect 14 or aspect 15, wherein the source of negative pressure is a vacuum pump.

Aspect 17: The oral appliance of aspect 14 or aspect 15, wherein the source of negative pressure is a micropump.

Aspect 18: The oral appliance of any one of aspects 14-17, wherein the source of negative pressure is configured to generate a pressure of at least about −5 cm H₂O.

Aspect 19: The oral appliance of any one of aspects 14-17, wherein the source of negative pressure is configured to generate a pressure between about −5 cm H₂O to about −20 cm H₂O.

Aspect 20: The oral appliance of any one of aspects 14-17, wherein the source of negative pressure is configured to generate a pressure between about −5 cm H₂O to about −10 cm H₂O.

Aspect 21: A method for treating a sleep disorder (e.g., a sleep disorder associated with mouth physiology such as obstructive sleep apnea, mouth breathing, snoring, teeth grinding, xerostomia, halitosis, hyper-salivation, tardive dyskinesia. and the like) in a subject, the method comprising stabilizing the tongue in a superior direction toward at least a portion of the subject's hard palate.

Aspect 22: The method of aspect 21, comprising forming a non-permanent attachment of the tongue to a surface of an oral appliance, the surface of the oral appliance comprising a wet-tolerant adhesive.

Aspect 23: The method of aspect 21, comprising applying negative pressure to the subject's oral cavity in a direction generally perpendicular to at least a portion of a dorsal surface of the subject's tongue, thereby stabilizing the tongue in a superior direction.

Aspect 24: The method of aspect 23, wherein negative pressure is applied to the subject's oral cavity in a direction generally perpendicular to a medial region of the dorsal surface of the subject's tongue.

Aspect 25: The method of aspect 23, wherein negative pressure is applied to the subject's oral cavity in a direction generally perpendicular to an anterior region of the dorsal surface of the subject's tongue.

Aspect 26: The method of any one of aspects 21-25, wherein the subject's tongue remains inside the subject's oral cavity.

Aspect 27: The method of any one of aspects 23-26, wherein the applied negative pressure does not result in substantial posterior movement of the subject's tongue toward the back of the subject's oral cavity.

Aspect 28: The method of any one of aspects 23-27, wherein the applied negative pressure promotes nasal breathing.

Aspect 29: The method of any one of aspects 23-28, wherein negative pressure is applied to a plurality of positions on the dorsal surface of the subject's tongue.

Aspect 30: The method of any one of aspects 23-29, wherein the negative pressure applied to the subject's oral cavity is at least about −5 cm H₂O.

Aspect 31: The method of any one of aspects 23-30, wherein the negative pressure applied to the subject's oral cavity is from about −5 cm H₂O to about −20 cm H₂O.

Aspect 32: The method of any one of aspects 23-31, wherein the negative pressure applied to the subject's oral cavity is from about −5 cm H₂O to about −10 cm H₂O.

Aspect 33: The method of any one of aspects 23-32, wherein the negative pressure is applied to the subject's oral cavity through an oral appliance configured to engage at least a portion of the dorsal surface of the subject's tongue in response to application of negative pressure.

Aspect 34: The method of aspect 33, wherein the oral appliance conforms to at least one upper tooth of the subject.

Aspect 35: The method of aspect 33 or aspect 34, wherein the oral appliance conforms to at least a portion of the subject's hard palate.

Aspect 36: The method of any one of aspects 23-35, wherein the negative pressure is applied to the subject's oral cavity through the oral appliance of any one of aspects 1-20.

Those skilled in the art will recognize, or be able to ascertain using no more than routine experimentation, many equivalents to the specific embodiments of the appliance and method described herein. Such equivalents are intended to be encompassed by the following claims.

What is claimed is:

1. An oral appliance for treating a sleep disorder in a subject, the oral appliance comprising:
a) a U-shaped element having an outer side wall, an inner side wall, and at least one biting surface, the outer side wall, the inner side wall, and the at least one biting surface together defining at least one channel configured to receive at least one upper tooth of the subject;
b) a palatal overlay element contiguous with the U-shaped element and configured to conform to at least a portion of the subject's hard palate, the palatal overlay element having an interior surface and an exterior surface and defining a plurality of perforations extending through the palatal overlay element between the interior and exterior surfaces;
c) a port in fluid communication with the plurality of perforations of the palatal overlay element, wherein the port is configured to communicate negative pressure to the plurality of perforations of the palatal overlay element;
wherein the exterior surface of the palatal overlay element is configured to engage at least a portion of a dorsal surface of the subject's tongue in response to application of negative pressure through the plurality of perforations of the palatal overlay element.

2. The oral appliance of claim 1, wherein the U-shaped element and the palatal overlay element are a monolithic body.

3. The oral appliance of claim 1, wherein the U-shaped element and the palatal overlay element are formed in the shape of a mold taken of at least one upper tooth of the subject and at least a portion of the subject's hard palate.

4. The oral appliance of claim 1, wherein at least one of the U-shaped element or the palatal overlay element comprises a thermoplastic, a silicone, an acrylic, a tacky polymer, a wet-tolerant adhesive, or a combination thereof

5. The oral appliance of claim 1, wherein the number of perforations is more than two.

6. The oral appliance of claim 1, wherein the number of perforations ranges from two to twenty-four.

7. The oral appliance of claim 1, wherein the number of perforations ranges from four to eight.

8. The oral appliance of claim 1, wherein the number of perforations ranges from four to six.

9. The oral appliance of claim 1, wherein the number of perforations is six.

10. The oral appliance of claim 1, wherein the port is positioned near a posterior region of the oral appliance.

11. The oral appliance of claim 1, wherein the port is positioned near an anterior region of the oral appliance.

12. The oral appliance of claim 1, wherein the port is positioned near a molar tooth of the subject when the oral appliance is inserted into the subject's oral cavity.

13. The oral appliance of claim 1, wherein the port is positioned near an incisor tooth of the subject when the oral appliance is inserted into the subject's oral cavity.

14. The oral appliance of claim 1, further comprising a source of negative pressure in fluid communication with the port.

15. The oral appliance of claim 14, further comprising a tube that provides fluid communication between the source of negative pressure and the port.

16. The oral appliance of claim 14, wherein the source of negative pressure is a vacuum pump.

17. The oral appliance of claim 14, wherein the source of negative pressure is a micropump.

18. The oral appliance of claim 14, wherein the source of negative pressure is configured to generate a pressure of at least about −5 cm H₂O.

19. The oral appliance of claim 14, wherein the source of negative pressure is configured to generate a pressure between about −5 cm H₂O to about −20 cm H₂O.

20. The oral appliance of claim 14, wherein the source of negative pressure is configured to generate a pressure between about −5 cm H₂O to about −10 cm H₂O.

21. A method for treating a sleep disorder in a subject, the method comprising stabilizing the tongue in a superior direction toward at least a portion of the subject's hard palate.

22. The method of claim 21, comprising forming a non-permanent attachment of the tongue to a surface of an oral appliance, the surface of the oral appliance comprising a wet-tolerant adhesive.

23. The method of claim 21, comprising applying negative pressure to the subject's oral cavity in a direction generally perpendicular to at least a portion of a dorsal surface of the subject's tongue, thereby stabilizing the tongue in a superior direction.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)
